(19) United States
(12) Patent Application Publication
(10) Pub. No.: US 2024/0122782 A1
(43) Pub. Date: Apr. 18, 2024

(54) VISION TRAINING DEVICE AND VISION TRAINING INSTRUMENT CROSS-REFERENCE TO RELATED APPLICATIONS

(71) Applicant: SUZHOU XUANJIA OPTICS AND ELECTRONICS TECHNOLOGY CO., LTD., Suzhou, Jiangsu (CN)

(72) Inventors: Peiwen LI, Suzhou (CN); Zhiming YAO, Suzhou (CN)

(21) Appl. No.: 18/397,754

(22) Filed: Dec. 27, 2023

Related U.S. Application Data
(63) Continuation of application No. PCT/CN2021/139015, filed on Dec. 17, 2021.

(30) Foreign Application Priority Data
Nov. 29, 2021 (CN) 202111433741.5

(51) Int. Cl. A61H 5/00

(52) U.S. Cl. CPC A61H 5/00; A61H 2201/0188; A61H 2201/0192; A61H 2201/10; A61H 2201/5007; A61H 2201/5092

(57) ABSTRACT

Disclosed is a vision training device. The vision training device includes: a treatment unit configured to generate treatment light for irradiating eyes of a user, an adjustment unit configured to adjust a position of the treatment unit, an imaging unit configured to image the eyes of the user, and a control module configured to receive data information generated by an analysis module. According to the vision training device, the imaging unit is arranged to shoot a position image of the eyes of the user, then a position of the eyes is converted into digital coordinate information, and a motor drives a gear and a toothed plate to mesh with each other, thereby achieving an effect of automatically adjusting a lens barrel, improving adjustment precision, improving adjustment efficiency, and achieving higher convenience.

### CROSS-REFERENCE TO RELATED APPLICATIONS

This application is a continuation of International Application No. PCT/CN2021/139015, filed on Dec. 17, 2021, which claims priority to Chinese Patent Application No. CN202111433741.5, filed on Nov. 29, 2021. All of the aforementioned applications are incorporated herein by reference in their entireties.

### TECHNICAL FIELD

The present application relates to the technical field of medical care apparatuses, in particular to a vision training device and a vision training instrument.

### BACKGROUND

With continuous advances in technology, mobile phones, computers and other electronic products are becoming more prevalent, and adolescents and children addicted to the electronic products are on the increase, causing their vision to continuously decline. Some studies show that the incidence rate of myopia of the adolescents and children is increasing year by year, and the onset age of myopia declines, that is, more and more young children suffer from myopia.

In order to relieve myopia symptoms, and improve vision, some patients use vision therapy apparatuses or low-intensity laser therapy. The vision therapy apparatuses or low-intensity laser therapy stimulates fundus cells of users by emitting to the users light with a certain wavelength, so as to improve vision. However, when eyes of patients are irradiated by light, pupils contract to vary greatly in diameter. The sizes of the pupils directly affect the energy of light entering the fundi of the users.

On account of certain differences in the pupil position and pupil distance of the eyes of the users using the apparatus each time, the position of the pupils that the laser light irradiates has a deviation. Besides, many users still rely on manual adjustment currently, which has certain errors and is of imprecision and low efficiency. To this end, a vision training device and a vision training instrument are proposed to solve the above problems.

In the prior art, as disclosed in Chinese Patent Application Publication CN1903151A, a semiconductor laser amblyopia treatment instrument includes a control unit, a voltage regulation circuit, a current regulation circuit, a timer and a semiconductor laser. The control unit is connected to the voltage regulation circuit, the current regulation circuit and the timer, and the semiconductor laser is connected to the voltage regulation circuit, the current regulation circuit and a timing switch K in series. The present application overcomes the defects of poor treatment effects and poor safety of an existing treatment instrument, and achieves the advantages of small size, light weight, portability, use safety, short treatment time and desirable treatment effects.

As disclosed in Chinese Patent Application Publication CN208741772U, an ophthalmic treatment instrument includes a base as a support portion of the entire instrument; a control unit configured to control operation of the entire instrument and including a signal input unit and an information feedback unit; a light source treatment unit including an eyepiece and a light source generation unit arranged behind the eyepiece; and an Internet of Things unit that can be connected to an upper computer for wireless or wired communication. The utility model induces cells on a retina to produce and release dopamine by controlling a wavelength, power, a time interval and a spatial area of light irradiated on the retina, so as to inhibit development of myopia and amblyopia and achieve the treatment purpose. The utility model further includes the control unit and the Internet of Things unit for treatment of myopia and amblyopia through remote control. A circuit principle and a mechanical structure for treating eyes by controlling light source emission are known.

As disclosed in Chinese Patent Application Publication CN207005779U, alight source module includes a light source circuit board and a light source shade, where a laser light emitter, a first light source and a second light source are arranged on the light source circuit board, and the first light source and the second light source are arranged around the laser light emitter; and the light source shade includes a shade shell and a connection barrel, the connection barrel is located at an end of the shade shell, the shade shell and the connection barrel are internally hollowed, and the shade shell covers and fixes the light source circuit board. The light source module according to the utility model is employed in a vision improvement device, and serves as an important product component for improving vision of a user. In the prior art, the principle of treating myopia and amblyopia with red light is accepted as a conventional treatment method. The applicant finds, through further study, that a treatment effect of myopia or amblyopia is influenced by alight flux entering fundi through pupils.

As disclosed in Chinese Patent Application Publication CN 209075129U of the applicant, a pupil distance adjustment mechanism for an ophthalmic medical apparatus includes a first frame body and a second frame body, where the second frame body is provided with a clamp portion capable of clamping an object, aside of the first frame body is provided with a gear adjustment mechanism, the gear adjustment mechanism at least includes a cantilever frame, a first gear, a second gear, a first adjustment handwheel and a second adjustment handwheel, the first gear correspondingly meshes with the first adjustment handwheel for gear transmission, and the second gear correspondingly meshes with the second adjustment handwheel for gear transmission. The first adjustment handwheel and the second adjustment handwheel can rotate around a joint between the first adjustment handwheel and the cantilever frame and a joint between the second adjustment handwheel and the cantilever frame respectively. The utility model has a simple structure, and can achieve self-locking positioning through meshing friction between the gears in the gear adjustment mechanism without adding other positioning devices, thus simplifying the structure. The utility model aims to adjust the distance between vision barrels manually, so as to adjust the coaxiality of a laser and the pupils, and allow the light to be irradiated on the fundi through the pupils to the maximum extent.

Through further study, by the applicant found that automatic adjustment can be implemented by combining an electric adjustment mechanism with an artificial intelligence recognition algorithm for a pupil image, and to this end, study is performed in the present application.

### SUMMARY

In order to solve the defects in the prior art, the present application provides a vision training device and a vision training instrument, which solve the problems of low efficiency and imprecision of manual adjustment of a distance between vision barrels, and great pupil contraction of a user during use of the apparatus.

In order to achieve the above objective, the present application provides the following technical solution:

A vision training device includes:
a treatment unit for generating treatment light;
an adjustment unit for adjusting a position of the treatment unit;
an imaging unit for imaging eyes of a user; where
the imaging unit includes:
a light splitting device configured to project, through refraction or reflection, an eye image mapped thereon on a shooting device,
the shooting device configured to take a picture of the eye image projected thereon,
and
an analysis module configured to receive the picture shot by the shooting device and analyze data information of pupils; and
a control module configured to receive the data information generated by the analysis module and control the adjustment unit to adjust the position of the treatment unit.

Preferably, the treatment unit is located at a side of the imaging unit.

Preferably, the adjustment unit is connected to the imaging unit, and drives the imaging unit to move to adjust the position of the treatment unit.

Preferably, the vision training device further includes a vision barrel, the vision barrel being connected to the imaging unit.

Preferably, the adjustment unit is connected to the vision barrel, and drives the vision barrel to move to adjust the position of the treatment unit.

Preferably, the treatment unit includes a light source module, the light source module including:
a laser device configured to generate laser light for treating myopia or amblyopia.

Preferably, the treatment unit further includes a visual target device configured to generate visible light or a pattern.

Preferably, the treatment unit further includes a mounting seat, and the light source module is located in the mounting seat.

Preferably, the mounting seat is a ball socket, the ball socket is arranged at a side of a vision barrel, a side of the vision barrel is provided with an open port, a cambered-shaped outer surface of the ball socket adaptively moves in the open port, and the ball socket may rotate in the open port.

Preferably, the treatment unit further includes a mounting seat, and the light source module is located in the mounting seat; and
the mounting seat is provided with several studs;
the light source module includes a circuit board, the laser is arranged on the circuit board, the circuit board is provided with several mounting holes, and the mounting holes are in adaptive connection to the studs; and
an outer side of the stud is provided with a spring, a screw is mounted in the stud, and as the screw is screwed out or screwed into a hole of the stud, a distance between the circuit board and the mounting seat increases or decreases accordingly.

Preferably, the vision training device further includes a locking cover, and the locking cover is configured to limit a position of a ball socket.

Preferably, the visual target device includes a plurality of visual target light sources arranged at the periphery of the laser device.

Preferably, a light spreading plate is arranged in front of the visual target light source.

Preferably, a light source emitted by the visual target light source has a wavelength within 380 nm-420 nm.

Preferably, the laser device includes a laser, and the laser emits and irradiates, through the light splitting device, treatment laser light on the pupils of the eyes of the user.

Preferably, the shooting device includes a photoelectric sensor and a lens barrel, a lens is arranged in the lens barrel, and the photoelectric sensor is arranged on a first circuit board.

Preferably, the light splitting device includes a beam splitter, and the beam splitter is obliquely arranged below the shooting device.

Preferably, a surface, facing the eyes of the user, of the beam splitter is provided with a reflective film to reflect the eye image onto the imaging unit.

Preferably, the imaging unit further includes an illumination device configured to provide an illumination environment to illuminate the eyes of the user.

Preferably, the vision training device further includes a cover, and the illumination device is arranged in the cover.

Preferably, the adjustment unit includes a drive motor, the drive motor is connected to a transmission assembly, the transmission assembly is connected to a guide mechanism, and the drive motor operates to drive the transmission assembly to move along the guide mechanism.

Preferably, the transmission assembly includes several gears and a rack that mesh with each other for transmission.

Preferably, the transmission assembly includes a drive gear, a toothed disc and a toothed plate, the drive gear meshes with the toothed disc, the toothed disc is provided with a pinion, the pinion meshes with the toothed plate, and the toothed plate is provided with a connection seat.

Preferably, the guide mechanism includes a slidable rod, two ends of the slidable rod are provided with stop plates, and the connection seat is in slidable connection to the slidable rod.

Preferably, the control module may further control operation of the treatment unit, operation of the imaging unit and operation of an illumination device.

Preferably, the imaging unit further includes a box, and a via hole suitable for light to penetrate therethrough is provided in the box.

Preferably, the analysis module analyzes and computes the shot eye picture in the following steps:
Step S1, preprocessing the eye picture obtained by the shooting device to obtain a picture with high black-and-white contrast;
Step S2, performing image denoising on the picture with high black-and-white contrast to obtain a denoised picture;
Step S3, processing the denoised picture with an outline recognition algorithm to obtain reference circle outline information and pupil outline information; and
Step S4, analyzing and computing center coordinates of a reference circle and center coordinates of the pupils according to the obtained reference circle outline information and the obtained pupil outline information.

Preferably, the position of the treatment unit is adjusted by the control module in the following steps:
establishing a spatial physical coordinate system, where a plane perpendicular to a direct direction of the eyes of the user is a coordinate plane, a center coordinate point of the pupils of the user is a coordinate origin, a vertical direction is an Y axis, and a horizontal direction is an X axis.
Step S5, turning on the visual target device in the treatment unit, and guiding the eyes of the user to gaze at the visual target device;
Step S6, turning on the illumination device, and simultaneously turning on the shooting device to shoot the pupils of the user and transmit picture information into the analysis module;
step S7: analyzing data of the picture, and determining whether the eyes of the user are open by the analysis module;
step S8: generating, under the condition that it is determined that the eyes of the user are not open completely, prompt information by the analysis module, to remind the user to open eyes wide, and re-executing S6;
continuing analyzing, under the condition that it is determined that the eyes of the user are open completely, a deviation value between the center coordinates of the reference circle and the coordinate origin by the analysis module, recording the deviation value as |Y|, and continuing to determine whether |Y| is smaller than a threshold X set by the system;
transmitting, under the condition that |Y| is greater than X, a signal to the control module by the analysis module, and controlling, by the control module, a motor in the adjustment unit to start until |Y| is not greater than X; and
step S9: emitting laser light by the treatment unit.

A vision training instrument includes any said vision training device above, where
the vision training instrument further includes a machine head and a base;
a pitch adjustment module configured to adjust a pitch angle of the machine head; and
a recognition device configured to recognize a distance of contact with the apparatus of a user;
a man-machine interaction module configured to display overall operation parameters of an apparatus.

Preferably, a front of the machine head is provided with an eye mask.

Preferably, the recognition device includes a fourth circuit board, a photoelectric receiver is connected to the fourth circuit board, a protective cover covers an outer surface of the photoelectric receiver, and the protective cover is arranged in a middle of an eye mask.

A hand-held vision training instrument includes any said vision training device above, and includes:
a housing, and the vision training device is arranged inside the housing.

Compared with the prior art, the present application provides the vision training device and the vision training instrument, which have the following beneficial effects:
1. According to the vision training device and a vision training instrument, the imaging unit is arranged to shoot a position image of the eyes of the user, then a position of the eyes of the user in the image is converted into digital coordinate information to be transmitted into the adjustment unit, and a motor in the adjustment unit drives a gear and a toothed plate to mesh with each other for relative transmission, thereby achieving an effect of automatically adjusting a lens barrel of some medical care apparatus, improving adjustment precision, improving adjustment efficiency, and achieving higher convenience compared with manual adjustment.
2. According to the vision training device and the vision training instrument, the vision barrel is arranged, such that the adjustment unit may directly adjust a position of the vision barrel to indirectly adjust and detect the imaging unit, which is conducive to placing of the adjustment unit and making full use of space in the housing.
3. According to the vision training device and the vision training instrument, the light source module is arranged, such that the eyes of user may be treated and trained with the laser light.
4. According to the vision training device and the vision training instrument, a vision direction of the user may be guided through the light or the pattern emitted by the visual target device, and the user is provided with a target direction, such that the eyes of the user may not roll at will, in this way, the light emitted by the laser is guaranteed to be directly face the pupils of the user, and the laser light may irradiate the fundi of the user to the maximum extent.
5. According to the vision training device and the vision training instrument, the mounting seat is arranged, such that the light source module may be conveniently placed, adjusted and fixed, an angle of light emitted by the light source module may be adjusted, rotary adjustment is implemented, and coaxiality of the laser light and an eye axis may be guaranteed.
6. According to the vision training device and the vision training instrument, a constant wavelength range of 380 nm-420 nm is arranged to reduce pupil contraction caused by laser light stimulation in the case of facing of the laser light.
7. According to the vision training device and the vision training instrument, the beam splitter is arranged, the light of the eyes of the user may be reflected or refracted in different directions, such that the shooting device and the light source module are not in the same orientation, the treatment light and shooting light do not interfere with each other, and the problem of limited space during placement of the device is avoided.
8. According to the vision training device and the vision training instrument, the eyes of the user are illuminated by the arranged illumination device, such that the problem that the eyes of the user in a closed and dark environment may not be shot clearly by the shooting device during use is avoided.
9. According to the vision training device and the vision training instrument, the pitch adjustment module is used to adjust a use posture of a head of the user, such that the user may be at a comfortable angle when using the apparatus, and comfort of the user and applicability of the apparatus may be improved accordingly.
10. According to the vision training device and the vision training instrument, the man-machine interaction module is arranged, such that the parameters of the apparatus may be shown for the user, and the user may also control operation of the entire apparatus through the man-machine interaction module.

### BRIEF DESCRIPTION OF THE DRAWINGS

FIG. 1 is an overall schematic diagram of a vision training device according to the present application.

FIG. 2 is a schematic structural diagram of an imaging unit of a vision training device according to the present application.

FIG. 3 is a schematic diagram of a shooting device of a vision training device according to the present application.

FIG. 4 is a schematic diagram of a light source module of a first type of a vision training device according to the present application.

FIG. 5 is a schematic diagram of an adjustment unit of a vision training device according to the present application.

FIG. 6 is a schematic diagram of a position of a machine head of a vision training device according to the present application.

FIG. 7 is a schematic diagram of a recognition device of a vision training instrument according to the present application.

FIG. 8 is an overall schematic diagram of a vision training instrument according to the present application.

FIG. 9 is a schematic diagram of eye coordinate positions in a vision training device and a vision training instrument according to the present application.

FIG. 10 is a schematic diagram of a connection mode between a mounting seat and a box according to a first embodiment of Example 3 of the present application.

FIG. 11 is a schematic diagram for connection between an imaging unit and a laser device according to Example 4 of the present application.

FIG. 12 is a schematic structural diagram of a hand-held vision training instrument according to Example 6 of the present application.

FIG. 13 is a schematic diagram for connection between an adjustment unit and an imaging unit of a vision training device and a vision training instrument according to the present application.

FIG. 14 is a schematic diagram of a light source module of a vision training device according to the present application.

FIG. 15 is a schematic diagram of a connection mode between a mounting seat and a vision barrel according to a second embodiment of Example 3 of the present application.

FIG. 16 shows steps for analyzing and computing a shot eye picture according to the present application.

### DETAILED DESCRIPTIONS OF THE EMBODIMENTS

The technical solutions in examples of the present disclosure are clearly and completely described below with reference to accompanying drawings in the examples of the present application. Apparently, the described examples are merely some examples rather than all examples of the present application. All the other examples derived by those of ordinary skill in the art based on the examples of the present application without creative efforts shall fall within the protection scope of the present application.

### Example 1 a Vision Training Device

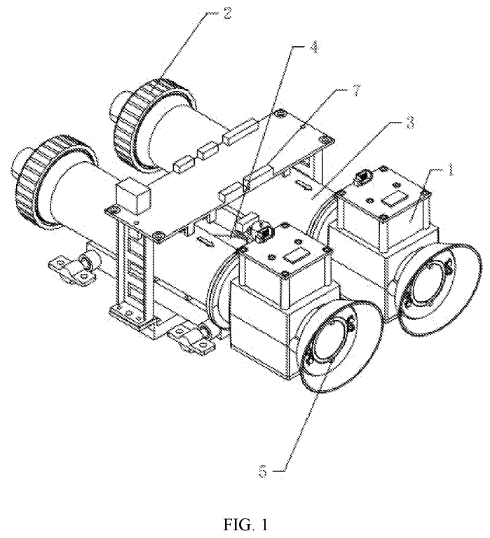

As shown in FIG. 1, a first type of a vision training device according to a first embodiment of the present application is provided.

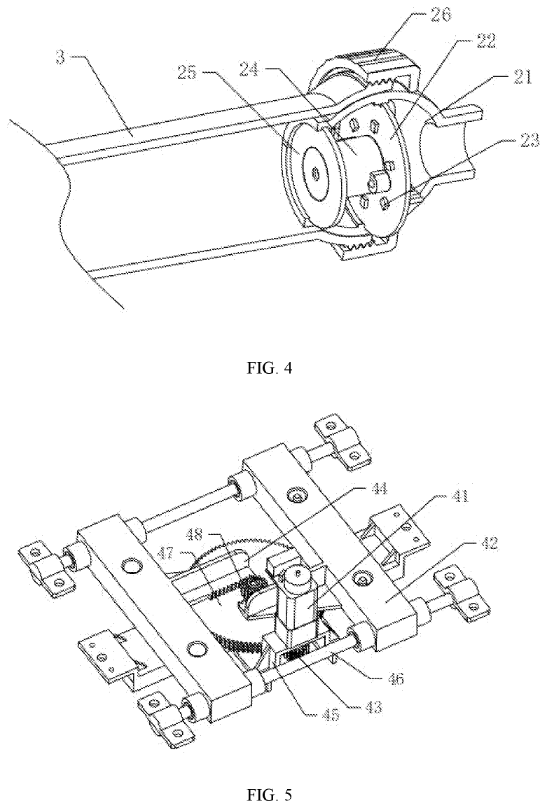

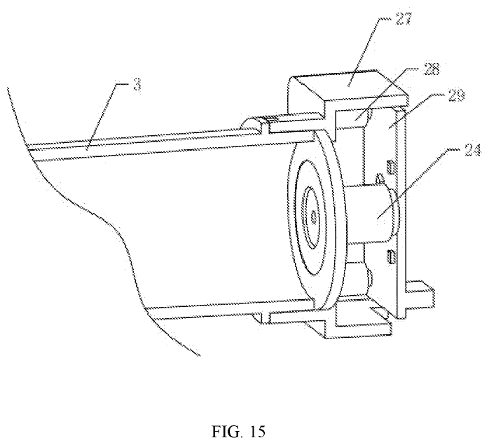

The vision training device includes a treatment unit 2 configured to generate treatment light for irradiating eyes of a user with reference to FIGS. 4 and 14. The treatment unit 2 includes alight source module, and the light source module includes:
a laser device configured to generate laser light for treating myopia or amblyopia, the treatment unit further includes a visual target device configured to generate visible light or a pattern, the treatment unit further includes a mounting seat, the light source module is located in the mounting seat, the mounting seat is a ball socket 21, the ball socket 21 is arranged at a side of a vision barrel 3, a side of the vision barrel 3 is provided with an open port, a cambered-shaped outer surface of the ball socket 21 adaptively moves in the open port, and the ball socket 21 may rotate in the open port. The ball socket includes a locking cover 26, and the locking cover 26 is configured to limit a position of the ball socket 21. The visual target device includes a plurality of visual target light sources 23 arranged at the periphery of a laser, a light spreading plate 25 is arranged in front of the visual target light source 23, and a light source emitted by the visual target light source 23 has a wavelength within 380 nm-420 nm. The laser device includes the laser 24, treatment light emitted by the laser 24 has a wavelength within 600 nm-700 nm, and output power is safety power of 5 mW or lower. The laser 24 emits and irradiates, through the light splitting device, the treatment laser light on the pupils of the eyes of the user. The laser device is mounted on a second circuit board 22, and is directly controlled by the second circuit board 22.

With reference to FIG. 5, the adjustment unit 4 is configured to adjust a position of the treatment unit 2. The adjustment unit 4 includes a drive motor 41, the drive motor 41 is connected to a transmission assembly, and the transmission assembly is connected to a guide mechanism. The drive motor 41 operates to drive the transmission assembly to move along the guide mechanism, the transmission assembly includes a drive gear 43, a toothed disc 47 and a toothed plate 44, the drive gear 43 meshes with the toothed disc 47, the toothed disc 47 is provided with a pinion 48, the pinion 48 meshes with the toothed plate 44, and the toothed plate 44 is provided with a connection seat 42. An indirect transmission mode of a plurality of gears is used, thereby avoiding the condition that direct drive by the motor 41 causes overload of the motor 41, and rotation delay accordingly. The guide mechanism includes a slidable rod 46, two ends of the slidable rod 46 are provided with stop plates, and the connection seat 42 is in slidable connection to the slidable rod 46. The entire connection seat 42 is connected to the box 104, such that when the adjustment unit 4 moves, the imaging unit 1 may be driven to move, and the entire treatment unit 2 may be driven to move in position by the imaging unit 1.

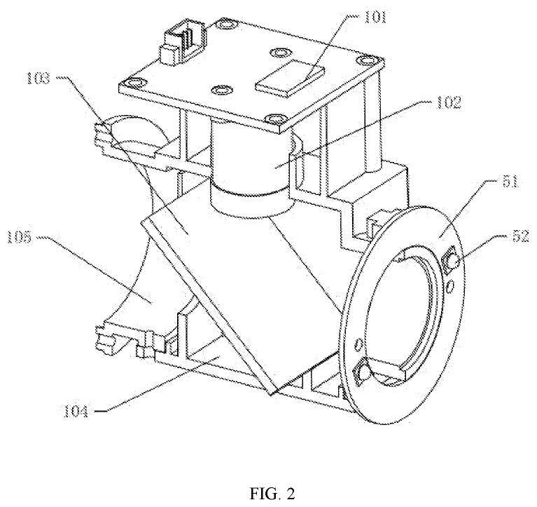

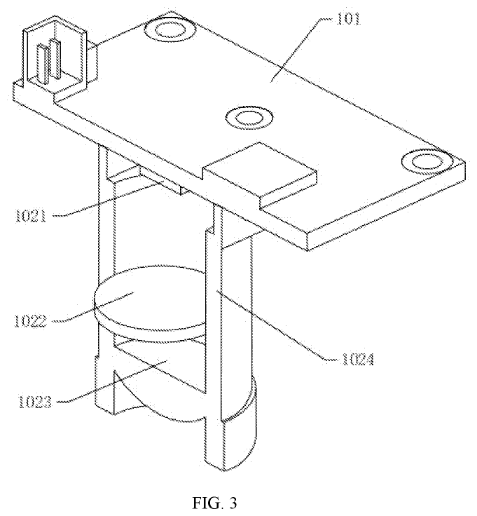

The imaging unit 1 is configured to image the eyes of the user with reference to FIGS. 2 and 3.

The imaging unit 1 includes:
the light splitting device configured to project, through refraction or reflection, an eye image mapped thereon on a shooting device 102, the light splitting device includes a beam splitter 103, and the beam splitter 103 is obliquely arranged below the shooting device 102.

The shooting device 102 is configured to take a picture of the eye image projected thereon, the shooting device 102 includes a photoelectric sensor 1021 and a lens barrel 1024, a lens 1023 is arranged in the lens barrel 1024, and the photoelectric sensor 1021 is arranged on a first circuit board 101. An optical filter 1022 is arranged, the optical filter 1022 is arranged between the photoelectric sensor 1021 and the lens 1023, so as to filter out surrounding stray light. The imaging unit further includes a box 104, and a via hole 105 suitable for light to penetrate therethrough is provided in the box 104.

A side of the box 104 is provided with a cover, and an illumination device 5 is arranged in the cover, and is configured to provide an illumination environment to illuminate the eyes of the user. The illumination device 5 includes a third circuit board 51, and the third circuit board 51 is connected to a lamp 52.

With reference to FIG. 1, an analysis module is configured to receive the picture shot by the shooting device 102 and analyze data information of pupils. In this example, the motor 41 in FIG. 5 may be controlled and adjusted to be started according to the data information of the pupils. The analysis module serves as a microcomputer or a processor on a circuit board 7 to perform program steps of analysis. For those skilled in the art, the analysis module and the control module are a set of software and hardware that may handle and execute programs. Without exclusion, the circuit board 7 may be divided into a plurality of portions, and each portion independently implements a specific function, such as independent control over the treatment unit 2, the illumination device 5 and the imaging unit 1.

The analysis module analyzes and computes the shot eye picture in the following steps:
Step S1: the eye picture obtained by the shooting device 102 is preprocessed to obtain a picture with high black-and-white contrast, image binarization is to set a gray value of a pixel on the image to 0 or 255, that is, the process of presenting the entire image with an obvious black-and-white effect. In short, a color pattern is converted into a black-and-white image. During digital image processing, the binarization of the image greatly reduces the amount of data in the image, thus highlighting an outline of a target.
Step S2: Image denoising is performed on the picture with high black-and-white contrast to obtain a denoised picture, median filtering may be used and is a nonlinear signal processing technology based on a sorting statistics theory that may effectively suppress noise, a basic principle of median filtering is to replace a value of a point in a digital image or digital sequence with a median value of points in a neighborhood of the point, such that surrounding pixel values are close to a real value, and an isolated noise point is eliminated.
Step S3: The denoised picture is processed with an outline recognition algorithm to obtain reference circle outline information and pupil outline information, and a reference circle outline is a circular outline of the lens barrel and is close to the pupil of the eye.

A curve is used to detect shapes such as a straight line, a circle, a parabola and an ellipse in an image, may be described by a certain functional relation, and has been successfully applied to many fields such as image analysis and pattern recognition. A basic principle of Hough transform is to transform a curve (including the straight line) in an image space into a parameter space, and determine description parameters of the curve by detecting extreme points in the parameter space, so as to extract a regular curve in the image.
Step S4: Center coordinates of a reference circle and center coordinates of the pupils are analyzed and computed according to the obtained reference circle outline information and the obtained pupil outline information, and numerical values of the center coordinates of the pupils are computed through a barycentric coordinate method.

The control module is configured to receive data information generated by the analysis module and control the adjustment unit 4 to adjust the position of the treatment unit 2. The control module may further control operation of the treatment unit 2, operation of the imaging unit 1 and operation of the illumination device 5. The control module is an integrated chip program on the circuit board 7, and the circuit board 7 is electrically connected to the analysis module of the imaging unit 1 and the motor 41 of the adjustment unit 4. In this example, the analysis module and the control module are on the same circuit board 7, but are not limited to the same circuit board 7, and may be arranged on different circuit boards respectively. The control module adjust a position of the imaging unit 1 in the following steps:

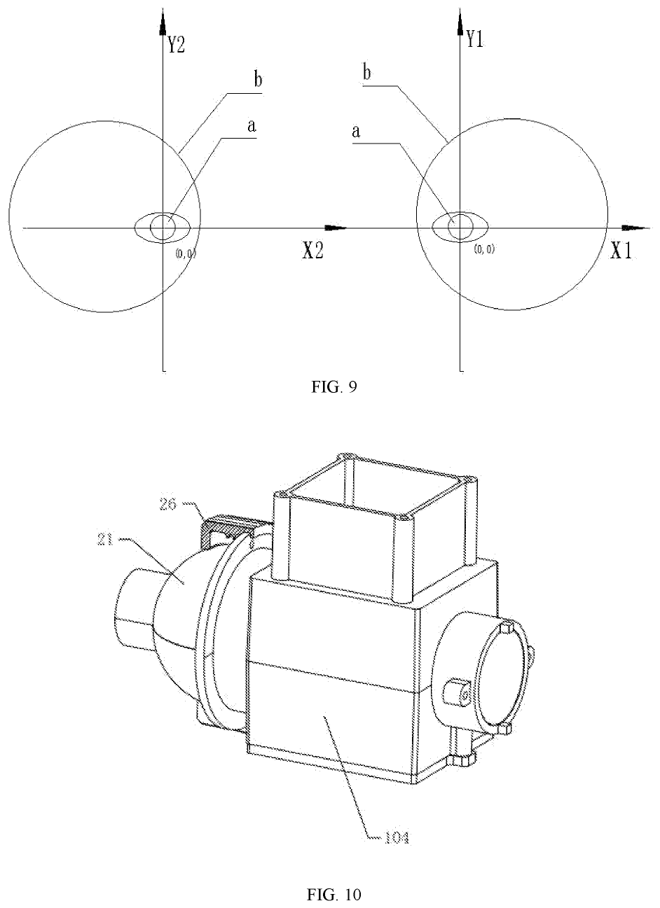

With reference to FIG. 9, a represents eyes, and b represents a reference circle. A spatial physical coordinate system is established, where a plane perpendicular to a direct direction of the eyes of the user is a coordinate plane, a center coordinate point of the pupils of the user is a coordinate origin, a vertical direction is an Y axis, and a horizontal direction is an X axis.
Step S5: The visual target device in the treatment unit is turned on, and the eyes of the user are guided to gaze at the visual target device.
Step S6: The illumination device 5 is turned on, and the shooting device 102 is simultaneously turned on to shoot the pupils of the user and transmit picture information into the analysis module.
Step S7: The analysis module may analyze data of the picture, and determine whether the eyes of the user are open.
Step S8: Under the condition that it is determined that the eyes of the user are not open completely, the analysis module generates prompt information, to remind the user to open eyes wide, and step S6 is re-executed.

Under the condition that it is determined that the eyes of the user are open completely, the analysis module continues analyzing a deviation value between the center coordinates of the reference circle and the coordinate origin, records the deviation value as |Y|, and continues to determine whether |Y| is smaller than a threshold X set by the system.

Under the condition that |Y| is greater than the threshold X, the control module may control the motor 41 in the adjustment unit 4 to start until |Y| is not greater than the threshold X. If a deviation between the eyes of the user in a vertical direction and a shooting center axis is too large, the deviation in the vertical direction is >±0.5 mm, a prompt may also be triggered to remind the user to move a head downwards or upwards until the pupils moves to a horizontal center coordinate axis for shooting of the imaging unit 1. Therefore, in a final shot eye picture, a vertical coordinate of the center coordinates of the pupils also tend to be 0, so it is only necessary to compare a difference between X coordinates of the center coordinates of the pupils and the shooting center coordinates, that is, the center coordinates of the reference circle, and the difference is recorded as the deviation value. For example, if the center coordinates of the reference circle is (5, 0.2), that is, a deviation value of the X axis is =5−0=5, an adjustment direction is relative, it is necessary to adjust the imaging unit 1 to move outwards by 5±0.5 mm, such that light irradiated by the light source module 2 directly irradiates the pupils. When the center coordinates of the reference circle is (−4, −0. 3), the deviation is =4−0=−4, it is necessary to move the imaging unit 1 inwards by 4±0.5 mm according to the same principle, and no analysis is required since a deviation in the vertical Y direction falls within the deviation range.
Step S9: The treatment unit 2 emits laser light, and a pupil distance of the device is automatically adjusted by connecting the analysis module, the control module and the adjustment unit 4 in series, such that applicability to different groups with different pupil distances may be achieved, and the conditions of low efficiency and large calibration errors of manual adjustment may be avoided.

### Example 2 a Vision Training Device Including a Vision Barrel

With reference to FIGS. 1 and 4, FIG. 4 is a partial sectional view of a treatment unit 2. A vision training device according to a first embodiment of the present application further includes a vision barrel 3, the vision barrel 3 is connected to an imaging unit 1, and an adjustment unit 4 is connected to the vision barrel 3, and drives the vision barrel 3 to move to adjust a position of the treatment unit 2. The treatment unit 2 may generate semiconductor laser light with a treatment effect, and the laser light may penetrate the vision barrel 3 to irradiate pupil positions of eyes. The imaging unit 1 is located above a side of the vision barrel 3, and may take a picture of the eyes. In FIG. 5, a connection seat 42 in the adjustment unit 4 is connected to the vision barrel 3 through bolts, such that the vision barrel 3 and the connection seat 42 are relatively fixed. A motor 41 is started to drive the connection seat 42 to move along a slidable rod 46 through meshing transmission of several gears and a rack. Since the vision barrel 3 and the connection seat 42 are relatively fixedly connected to each other, the vision barrel 3 and the connection seat 42 move synchronously, and the treatment unit 2 is fixed on the vision barrel 3, so the treatment unit 2 moves indirectly. The entire motor 41 and a circuit board 7 are connected to a bottom plate 45, the bottom plate 45 supports the motor 41 and the circuit board 7, and the bottom plate 45 is connected in an entire housing to fix the entire device.

According to analysis on a pupil picture by an analysis module, the position of the treatment unit 2 is automatically adjusted through a method for adjusting and controlling the motor 41 in Example 1, so as to increase the amount of light that a light source module irradiates the pupil positions of the user into fundi through the vision barrel 3, stimulate blood flow and thicken choroid of the fundi.

### Example 3 a Vision Training Device Including a Mounting Seat

With reference to FIGS. 4 and 10, a first type of first vision training device including the mounting seat is shown, FIG. 4 is a partial sectional view of a treatment unit 2, and is used to show a schematic diagram of an inside of a ball socket 21. In this example, the mounting seat located at a side of a box 104 is a ball socket 21, and the box 104 is used for mounting the imaging unit 1. As shown in FIG. 4, a cavity is provided inside the ball socket 21, and a light source module is located in the cavity of the ball socket 21. The ball socket 21 further includes a locking cover 26, and the locking cover 26 is used to limit a position of the ball socket 21. The spherical ball socket 21 is located in a spherical cavity at an end face of the box 104, and may cooperatively move to adjust a direction of laser light emitted by the light source module, and the locking cover 26 fixes the light source module in position. When an angle of treatment light is to be adjusted, an angle of emitted light may be adjusted by directly rotating the ball socket 21.

With reference to FIG. 15, a second type of vision training device including a mounting seat includes a mounting seat housing 27. Studs 28 are arranged at the periphery of the mounting seat housing 27, a circuit board 28 is adaptively arranged on the mounting seat housing 27, positioning holes 29 are provided at the periphery of the circuit board 28, and the positioning holes 29 may be correspondingly connected to the studs 28. The studs 28 are provided with holes, and the periphery of the circuit board 28 is fixed on the studs 28 with screws. A spring sleeves each stud 28, one end of the spring abuts against a surface of the circuit board 28, and the other end abuts against an inner side surface of the mounting seat housing 27. When the circuit board 28 moves in a direction of the stud 28 through the positioning hole 29, the circuit board may be under action of the spring. By screwing in or screwing out the screws at different positions of the circuit board 28 relative to the studs, positions of the circuit board 28 at different positions relative to the inner side surface of the mounting seat housing 27 may be adjusted, and then an emission angle of a laser 24 arranged at a center of the circuit board 28 may be adjusted, which is particularly suitable for keeping a coincidence state of the light emitted by the laser 24 along a central axis of a vision barrel 3.

### Example 4 a Vision Training Device Having a Light Source Module Fixed at a Side of a Box

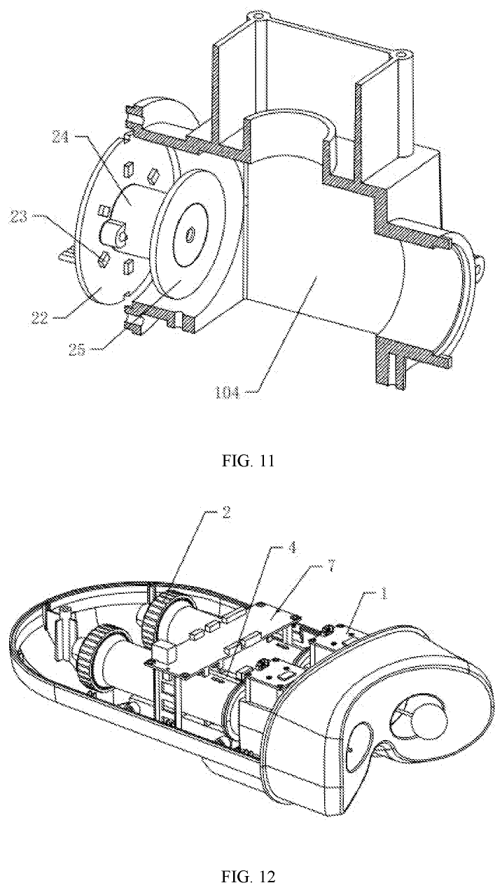

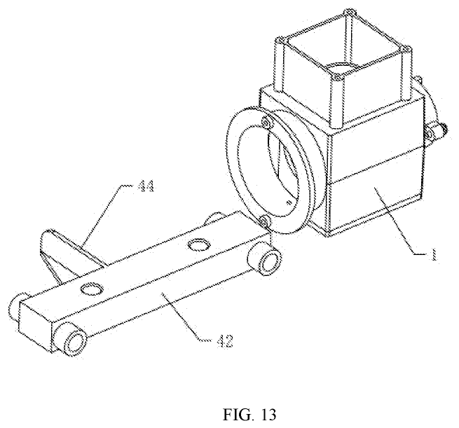

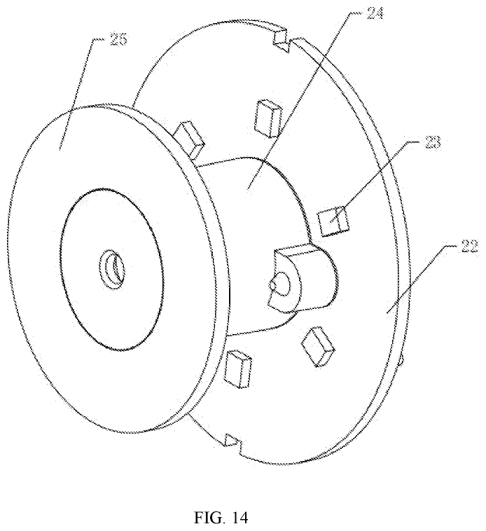

With reference to FIG. 11, the light source module is located in a cavity of the box 104, and is fixed in position directly by bolts. According to an embodiment of the present application, it is necessary to adjust a relative position between an emitted laser beam of semiconductor laser light with a treatment effect and pupils of human eyes. So, the laser beam may be caused to directly irradiate pupil positions as much as possible, and the amount of light entering the pupils is increased. In the embodiment of the present application, an imaging unit 1 is used to image eye positions, an imaged picture is analyzed, and a position of the treatment unit 2 is adjusted according to an analysis result, such that a light beam generated by the treatment unit 2 may directly irradiate the pupils.

### Example 5 a Vision Training Instrument

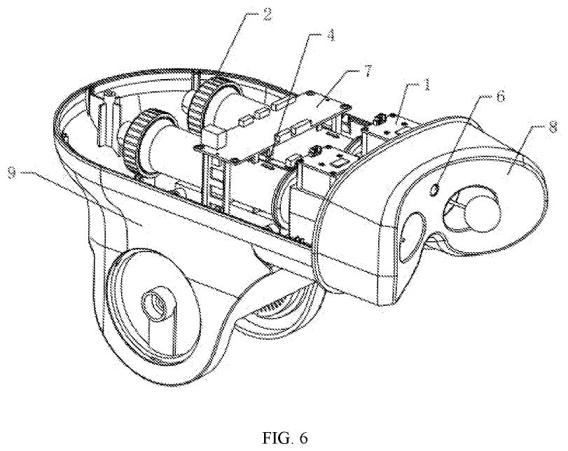

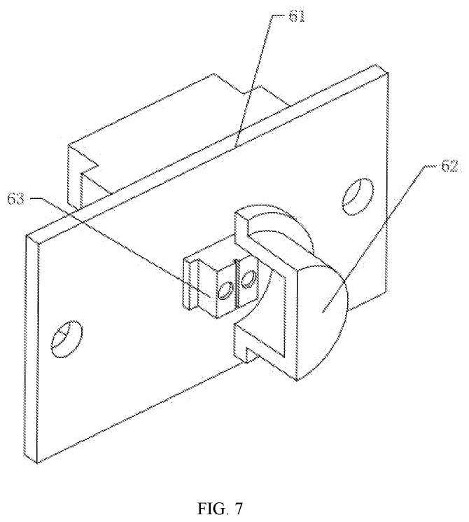

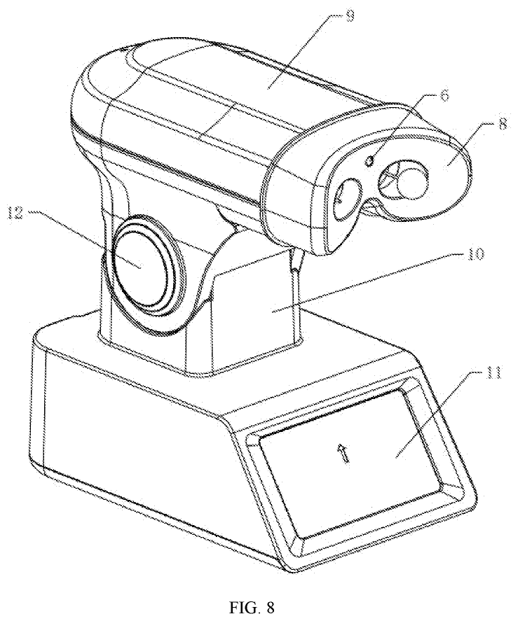

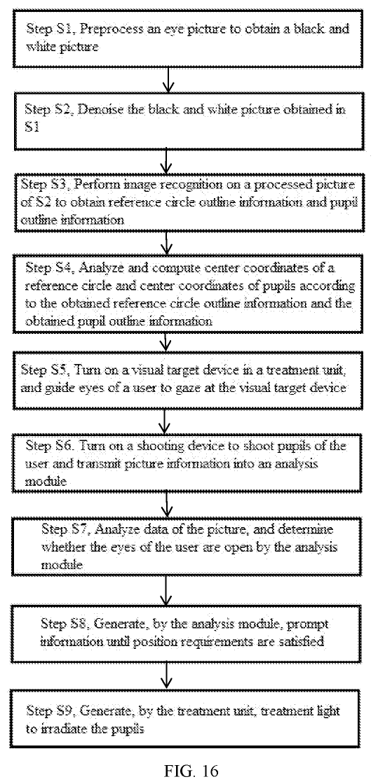

With reference to FIGS. 6-9 and FIG. 16, the vision training instrument includes any vision training device above, and further includes:
a machine head 9 and a base 10;
a pitch adjustment module 12 configured to adjust a pitch angle of the machine head 9;
a recognition device 6 configured to recognize a distance of contact with the apparatus of a user, the entire vision training instrument is provided with the pitch adjustment module 12, and when using the vision training instrument, the user may adjust a pitch angle of the machine head 9 according to comfort thereof, so as to improve the use comfort of the user; and
a man-machine interaction module 11 configured to display overall operation parameters of an apparatus, the man-machine interaction module 11 may provide an interaction interface between the user and the apparatus, and display all the parameters during operation of the apparatus, and the user may directly operate the interaction interface to control the overall operation of the apparatus in real time.

In this example, a front of the machine head 9 is provided with an eye mask 8, the eye mask 8 is made of rubber, the eye mask 8 is a position for contact with skin of the user, and the rubber material has certain flexibility, thus reducing irritation of metal and plastic to the skin of the user.

It shall be noted that the recognition device 6 includes a fourth circuit board 61, a photoelectric receiver 63 is connected to the fourth circuit board 61, a protective cover 62 covers an outer surface of the photoelectric receiver 63, and the protective cover 62 is arranged in the eye mask 8. Laser light is emitted from the recognition device 6 to the skin of the user, and is reflected and received, and a position distance is analyzed and determined through intensity of received reflected light, so as to remind the user whether an eye distance reaches a predetermined standard value. In this way, a more desirable treatment effect of the treatment apparatus is achieved during a treatment process, and when the preset value is achieved, the user directly blocks external ambient light, thereby avoiding interference caused by the external ambient light during the treatment process.

### Example 6 a Hand-Held Vision Training Instrument

With reference to FIG. 12, the hand-held vision training instrument includes any vision training device above, and further includes a housing, the housing is convenient to hold by a user. The vision training device is arranged inside the housing, and the hand-held vision training instrument has high portability and has a relatively wide range of application scenarios accordingly.

Electrical elements in the description are all electrically connected to an external main controller and 220 V mains electricity, and the main controller may be a conventional known control apparatus such as a computer.

Operation principle: when the user put eyes against an eye mask 8, a recognition device 6 emits laser light and then determines whether a distance of the user reaches a predetermined standard value through light reflected from skin of the user. If the user is not within the standard distance, a buzzer may sound to remind the user to get closer. When the user is within the standard distance, a visual target device may light up to provide a target for the user to gaze at, such that the user may gaze at the visual target device. Secondly, an illumination device 5 in an imaging unit 1 may illuminate the eyes of the user, and then a shooting device 102 may shoot the eyes of the user. Under the action of a single-layer reflective film arranged on a beam splitter 103, an eye image of the user may be mapped to a position below the shooting device 102, making it more convenient to shoot by the shooting device 102. After shooting, data information is transmitted to an analysis module, and the analysis module may determine whether the eyes of the user are open, and remind the user in a voice prompt mode or other prompt mode to open eyes in the case that the eyes are not open, shooting is re-performed, and the steps above are performed in cycles until a clear picture of open eyes is taken. Then, the analysis module compares and analyzes pupil positions in the shot image with an established coordinate axis, and analyzes a deviation between the pupil positions and an established coordinate axis origin. When the deviation value i≤0.5 mm, the analysis module may transmit data to a control module. The control module turns on a laser 24 to emit treatment light to irradiate the eyes of the user. When the deviation value i>0. 5 mm, the analysis module transmits data to a control module, the control module computes the number of turns of a motor 41, and then transmits an electrical signal to the motor 41. The motor 41 may drive a toothed plate 44 to move, and directly drive a vision barrel 3 to move in position by using a transmission relation between gears based on the received signal and specified rotation turns, finally, the imaging unit 1 is driven to move to achieve adjustment of a horizontal position, and a vertical adjustment mode may be achieved by the user moving a head up and down. In this technical solution, apparatus adjustment for the pupil distance is achieved only through the horizontal adjustment. After the apparatus moves to the standard value, the shooting device 102 may repeat the shooting step above to re-perform shooting, computation and analysis in cycles until a position of the user is within a fixed value range. The treatment laser light may directly irradiate fundi of the eyes of the user through the beam splitter 103 for treatment training. The entire vision training instrument is equipped with a pitch adjustment module 12, and during use, the user may adjust a position of a machine head 9 according to comfort, thereby improving use comfort of the user. A man-machine interaction module 11 may provide a communication interface between the user and the apparatus, and display all the parameters during operation of the apparatus, and the user may directly operate an interactive interface to control the overall operation of the apparatus in real time.

It should be noted that in the description, relational terms such as first and second are merely used to distinguish one entity or one operation from another entity or another operation, and do not necessarily require or imply any such actual relationship or order among these entities or operations. In addition, the terms “comprise”, “include”, “encompass” or any other variant thereof are intended to cover non-exclusive encompassing, such that a process, a method, an article or an apparatus including a series of elements not only includes those elements, but includes other elements not listed clearly, or further includes elements inherent to such process, method, article or apparatus. In the case of no more limitation, an element limited by the phrase “including a . . . ” does not exclude another same element existing in a process, method, article or apparatus including the element.

1. A vision training device, comprising:
a treatment unit configured to generate a treatment light;
an adjustment unit configured to adjust a position of the treatment unit;
an imaging unit configured to image eyes of a user;
and
a control module configured to receive data information and control the adjustment unit to adjust the position of the treatment unit;
wherein the imaging unit comprises:
a shooting device configured to take a picture of the eye image projected thereon;
a light splitting device configured to project, through refraction or reflection, an eye image mapped thereon on the shooting device, and
an analysis module configured to receive the picture shot by the shooting device and analyze the data information of pupils transmitted to the control module.

2. The vision training device according to claim 1, wherein the analysis module analyzes and computes the shot eye picture in the following steps:
step S1, preprocessing the eye picture obtained by the shooting device to obtain a picture with high black-and-white contrast;
step S2, performing image denoising on the picture with high black-and-white contrast to obtain a denoised picture;
step S3, processing the denoised picture with an outline recognition algorithm to obtain reference circle outline information and pupil outline information; and
step S4, analyzing and computing center coordinates of a reference circle and center coordinates of the pupils according to the obtained reference circle outline information and the obtained pupil outline information; and
wherein the position of the treatment unit is adjusted in the following steps:
establishing a spatial physical coordinate system, wherein a plane perpendicular to a direct direction of the eyes of the user is a coordinate plane, a center coordinate point of the pupils of the user is a coordinate origin, a vertical direction is an Y axis, and a horizontal direction is an X axis;
step S5, turning on the shooting device to shoot the pupils of the user and transmit picture information into the analysis module;
step S6, analyzing, by the analysis module, a deviation value between the center coordinates of the reference circle and the coordinate origin, recording the deviation value as |Y|, and determining, by the analysis module, whether |Y| is greater than a threshold X set by the system;
transmitting, under the condition that |Y| is greater than the threshold X, a signal to the control module by the analysis module, and controlling, by the control module, a motor in the adjustment unit to start to adjust the position of the treatment unit until |Y| is not greater than the threshold X; and
step S7: emitting laser light by the treatment unit.

3. The vision training device according to claim 1, wherein
the adjustment unit is connected to the imaging unit, and drives the imaging unit to move to adjust the position of the treatment unit; and
the treatment unit is located at a side of the imaging unit.

4. The vision training device according to claim 3, further comprising:
a vision barrel, the vision barrel being connected to the imaging unit, wherein
the adjustment unit is connected to the vision barrel, and drives the vision barrel to move to adjust the position of the treatment unit.

5. The vision training device according to claim 1, wherein
the treatment unit comprises a light source module, the light source module comprising:
a laser device configured to generate laser light for treating myopia or amblyopia, the laser device comprising a laser, and the laser emitting and irradiating, through the light splitting device, treatment laser light on the pupils of the eyes of the user.

6. The vision training device according to claim 5, wherein the treatment unit further comprises a mounting seat, and the light source module is located in the mounting seat; and
the mounting seat is a ball socket, the ball socket is arranged at a side of a vision barrel, a side of the vision barrel is provided with an open port, a cambered-shaped outer surface of the ball socket adaptively moves in the open port, and the ball socket rotates in the open port.

7. The vision training device according to claim 5, wherein the treatment unit further comprises a mounting seat, and the light source module is located in the mounting seat;
the mounting seat is provided with several studs;
the light source module comprises a circuit board, the laser is arranged on the circuit board, the circuit board is provided with several mounting holes, and the mounting holes are in adaptive connection to the studs; and
an outer side of the stud is provided with a spring, a screw is mounted in the stud, and as the screw is screwed out or screwed into a hole of the stud, a distance between the circuit board and the mounting seat increases or decreases accordingly.

8. The vision training device according to claim 1, wherein the light splitting device comprises a beam splitter, the beam splitter is obliquely arranged below the shooting device, a surface, facing the eyes of the user, of the beam splitter is provided with a reflective film to reflect the eye image onto the imaging unit.

9. The vision training device according to claim 1, wherein the shooting device comprises a photoelectric sensor and a lens barrel, a lens is arranged in the lens barrel, and the photoelectric sensor is arranged on a first circuit board.

10. The vision training device according to claim 1, wherein the imaging unit further comprises an illumination device configured to provide an illumination environment to illuminate the eyes of the user; and
the imaging unit further comprises a box adapted to the shooting device, a main portion of the shooting device is arranged in the box, a side of the box is provided with a cover, and the illumination device is arranged in the cover.

11. The vision training device according to claim 1, wherein
the adjustment unit comprises a drive motor, the drive motor is connected to a transmission assembly, the transmission assembly is connected to a guide mechanism, the transmission assembly comprises a drive gear, a toothed disc and a toothed plate, the drive gear meshes with the toothed disc, the toothed disc is provided with a pinion, the pinion meshes with the toothed plate, and the toothed plate is provided with a connection seat;
the guide mechanism comprises a slidable rod, and the connection seat is in slidable connection to the slidable rod; and
the drive motor operates to drive the transmission assembly to move along the guide mechanism, and a transmission mode of the transmission assembly uses meshing transmission of several gears and a rack.

12. The vision training device according to claim 1, wherein the control module further control operation of the treatment unit, operation of the imaging unit and operation of an illumination device.

13. The vision training device according to claim 5, wherein the treatment unit further comprises a visual target device configured to generate visible light or a pattern; and
the visual target device comprises a plurality of visual target light sources arranged at the periphery of the laser, a light spreading plate is arranged in front of the visual target light source, and a light source emitted by the visual target light source has a wavelength within 380 nm-420 nm.

14. The vision training device according to claim 5, wherein the laser light has a wavelength within 600 nm-700 nm, and output power of a laser terminal of the laser is less than 5 mW.

15. A vision training instrument, comprising the vision training device according to claim 1, wherein
the vision training instrument further comprises a machine head and a base;
a pitch adjustment module configured to adjust a pitch angle of the machine head; and
a man-machine interaction module configured to display overall operation parameters of an apparatus.

16. The vision training instrument according to claim 15, further comprising:
a vision training instrument recognition device configured to recognize a distance of contact with the apparatus of a user,
wherein the recognition device comprises a fourth circuit board, a photoelectric receiver is connected to the fourth circuit board, a protective cover covers an outer surface of the photoelectric receiver, and the protective cover is arranged in a middle of an eye mask.

17. A hand-held vision training instrument, comprising the vision training device according to claim 1, wherein
the hand-held vision training instrument comprises a housing, and the vision training device is arranged inside the housing.
